(19) United States
(12) Patent Application Publication
(10) Pub. No.: US 2019/0195977 A1
(43) Pub. Date: Jun. 27, 2019

(54) DETERMINING IMAGING QUALITY INFORMATION FOR A MAGNETIC RESONANCE IMAGING APPARATUS

(71) Applicant: Siemens Healthcare GmbH, Erlangen (DE)

(72) Inventors: Andre de Oliveira, Uttenreuth (DE); Martin Harder, Nürnberg (DE); Stephan Kannengießer, Wuppertal (DE); Wilfried Landschütz, Baiersdorf (DE)

(21) Appl. No.: 16/231,504

(22) Filed: Dec. 22, 2018

(30) Foreign Application Priority Data
Dec. 22, 2017 (EP) 17209923.6

(51) Int. Cl. G01R 33/56; A61B 5/055; A61B 6/00; G01R 33/32

(52) U.S. Cl. CPC G01R 33/5608; G01R 33/32; A61B 6/5217; A61B 5/055

(57) ABSTRACT

Noise information describing general noise properties of a magnetic resonance imaging apparatus (MRIA) in an imaging volume is measured using a predetermined first magnetic resonance sequence. Patient image data is acquired in at least one imaging region of a patient using a predetermined second magnetic resonance sequence. An anatomical landmark of a group of predetermined anatomical landmarks is localized in the patient image data by a landmark detection algorithm. For landmarks, reference information regarding imaging quality information is provided in a database. A landmark-specific signal-to-noise ratio is determined for each localized landmark from the patient image data at the landmark and the noise information, and the imaging quality information is determined from the landmark-specific signal-to-noise ratio. Assessment of the performance of the MRIA and/or the determination of the imaging parameters is performed depending on a comparison of the imaging quality information and the corresponding reference information in the database.

This application claims the benefit of EP 17209923.6, filed on Dec. 22, 2017, which is hereby incorporated by reference in its entirety.

### BACKGROUND

The present embodiments relate to determining imaging quality information for a magnetic resonance imaging apparatus.

A magnetic resonance imaging apparatus is a complex system composed of hundreds of subcomponents that are very sensitive to environmental changes. Therefore, the magnetic resonance imaging apparatus is to be constantly monitored to guarantee patient and staff safety as well as a high image quality. Typically, the components of a magnetic resonance imaging apparatus are to be tested periodically during quality assurance measurements to check whether the components are working within specified ranges. These quality assurance measurements require phantom measurements to provide that the results of the experiments are reproducible and comparable to previous measurements. However, during these phantom measurements, the magnetic resonance imaging apparatus is not available for patient examination. It is therefore desirable to reduce the number of such quality assurance measurements based on phantom measurements to increase the capacity utilisation of the magnetic resonance imaging apparatus.

US 2012/0010495 A1 describes the generation of magnetic resonance images of a volume section within an examination object via a magnetic resonance scanner, in which a number of quality inspections are performed on at least one magnetic resonance image. In the case of failed inspections, an action is automatically performed in order to improve a quality when generating more of the magnetic resonance images. The automatically performed quality inspection shortens a wait time of a patient in the magnetic resonance imaging apparatus. Based on the result of the inspection of the at least one magnetic resonance image, an action may be performed in order to improve the quality of magnetic resonance images generated after this action.

### SUMMARY AND DESCRIPTION

The scope of the present invention is defined solely by the appended claims and is not affected to any degree by the statements within this summary.

It would be desirable to not only improve the upcoming images of an examination procedure, but also to gain information about the performance of the magnetic resonance imaging apparatus in order to eliminate or reduce the number of service staff visits needed to provide that the system is operating according to a specification.

The present embodiments may obviate one or more of the drawbacks or limitations in the related art. For example, a method to obtain quality information during examination of different patients, where the quality information is not entirely patient-specific and therefore comparable over several examinations, is provided.

According to an embodiment, a method includes measuring a noise information describing general noise properties of a magnetic resonance imaging apparatus in an imaging volume using a predetermined first magnetic resonance sequence. Patient image data is acquired in at least one imaging region of the patient using a predetermined second magnetic resonance sequence. At least one anatomical landmark of a group of predetermined anatomical landmarks is localized in the patient image by a landmark detection algorithm. For each landmark and/or for at least one pair of landmarks, a reference information regarding the imaging quality information is provided in a database. A landmark-specific signal-to-noise ratio is determined for each localized landmark from the patient image data at the landmark and the noise information, and the imaging quality information is determined from the landmark-specific signal-to-noise ratio. The assessment of the performance of the magnetic resonance imaging apparatus and/or the determination of the imaging parameters is performed depending on a comparison of the imaging quality information and the corresponding reference information in the database.

One or more of the present embodiments are based on the insight that landmark-specific signal-to-noise ratios may be used to reduce individual variations of patient-specific measurements since a certain type of landmarks includes a certain type of tissue that has a defined range of relevant magnetic resonance imaging parameters like T1, T2, proton density, or other parameters. Using the same magnetic resonance sequence, the landmark will thus have similar signal intensities for different patients. Therefore, this tissue-specific or landmark-specific signal-to-noise ratio for a predetermined group of landmarks may be used as a reproducible measurement for the evaluation of the performance of the magnetic resonance imaging apparatus if the tissue-specific or landmark-specific signal-to-noise ratio is determined using the same magnetic resonance imaging sequences for all patients. Additionally or alternatively, these landmark-specific signal-to-noise ratios, as a measure of imaging quality, may enable an adaption or an optimization of imaging protocols.

The determination of the landmark-specific signal-to-noise ratio may, for example, be performed at least once for each patient (e.g., at the beginning of the examination process of the patient with the magnetic resonance imaging apparatus, as an integral part of the examination process, obviating the need for dedicated, separate quality assurance measurements). Since the fluctuation of tissue composition at the landmark between different patients is sufficiently low, at least for the group of landmarks for which a reference information has been stored in the database, comparison of values for different examinations and thus different patients is, for example, possible, as will be further described below. For example, the reference information may include or be derived from imaging quality information or comparison results of previous examinations. Thus, in embodiments, the reference information in the database may be updated depending on the current imaging quality information and/or comparison results.

It is one advantage of the method according the present embodiments that, based on the usage of landmark-specific signal-to-noise ratios, imaging quality information obtained for different patients is comparable and therefore permits the assessment of the magnetic resonance imaging apparatus performance, so that phantom measurements for quality assurance measurement may be eliminated or reduced in number.

A second aspect of the method is the usage of these landmark-specific signal-to-noise ratios for the determination of imaging parameters, which may be used, for example, for an adaption or for an optimization of an imaging protocol. For example, the imaging parameters may be determined for and used in sequences and/or protocols, which will be conducted subsequently in the on-going examination process. For example, a database of imaging parameters associated with certain ranges of landmark-specific signal-to-noise ratios may be provided. However, one or more of the present embodiments aim at quality assessment of the magnetic resonance imaging apparatus.

It is not required for the patient image to show every landmark for which a reference information is obtainable from the database. In many cases, only parts of the patient will be inside the imaging volume of the magnetic resonance apparatus, such that, for example, the database contains reference information for a group of landmarks essentially uniformly distributed over the body. In an embodiment, the landmark detection algorithm may detect available landmarks and check if these landmarks are also present in the database. For the detected at least one landmark for which a reference information is available, the landmark-specific signal-to-noise ratio is determined.

The determination of a signal-to-noise ratio requires at least two different values, as both information about the signal and information about the noise is used. The information about the noise is determined by measuring noise information that describes the general noise properties of the magnetic resonance imaging apparatus in the imaging volume. For that, the predetermined first magnetic resonance sequence is used to perform a noise measurement by recording a noise level present in the at least one coil element used for measuring, for example, one or more RX channels of a body coil. Such a noise measurement may be performed, for example, every time that a new coil configuration is selected or that a table of the magnetic resonance imaging apparatus, on which the patient is positioned, is moved.

The second value for the signal-to-noise ratio (e.g., the signal information) is determined by acquiring patient image data in the at least one imaging region of the patient using a predetermined second magnetic resonance sequence. Using the second magnetic resonance sequence, the patient image data in at least one imaging region of the patient is measured. In this patient image data, at least one anatomical landmark of a patient or a pair of anatomical landmarks of the patient may be localized by a landmark detection algorithm. At least one anatomical landmark of a group of predetermined anatomical landmarks available for the imaging region may be used. For each anatomical landmark or for each pair of anatomical landmarks, which is available for localization, a reference information regarding the imaging quality information is provided in a database. The database may be part of the magnetic resonance imaging apparatus or the database may be an external database that may be connected to the magnetic resonance imaging apparatus, for example, by a communication network, so that the database may be accessed by the magnetic resonance imaging apparatus or so that the magnetic resonance imaging apparatus may request information from the database.

For the extraction of the landmark from the patient image data, different methods and/or algorithms are known in the state of the art. For example, a landmark localization algorithm described by Zhou et al. (X. S. Zhou, Z. Peng, Y. Zhan, M. Dewan, B. Jian, A. Krishnan, Y. Tao, M. Harder, S. Grosskopf, and U. Feuerlein. Redundancy, redundancy, redundancy: the three keys to highly robust anatomical parsing in medical images. In MIR '10: Proc. Int'l Conf. Multimedia Info Retrieval, pages 175-184, New York, N.Y., USA, 2010) may be used in a method according to the present embodiments. When the at least one anatomical landmark or pair of anatomical landmarks is localized within the patient image data, a signal value describing the signal level at the landmark or at each landmark of a pair of landmarks may be extracted from the patient image data. From this signal level information, the landmark-specific signal-to-noise ratio for each localized landmark may be determined by, for example, dividing by the noise information.

It is insignificant whether the noise information is determined before the patient image data is acquired or if the acts are conducted vice versa. Hence, the acts of measuring the noise information and of acquiring the patient image data may be conducted in an arbitrary order.

The landmark-specific signal-to-noise ratio is used for the determination of the imaging quality information. As a consequence, also the imaging quality information is landmark-specific. To assess the performance of the magnetic resonance imaging apparatus and/or for a determination of the imaging parameters, the imaging quality information is compared to the corresponding reference information regarding the imaging quality information in the database. This comparison between the currently determined imaging quality information and the stored reference information enables the assessment of the performance of the magnetic resonance imaging apparatus and/or the determination of the imaging parameters.

The usage of a landmark-specific signal-to-noise ratio allows a comparison of patient image data collected for different patients, since properties of the landmark, like, for example, signal level from T1 decays, T2 decays, T2* decays, proton densities and others, as well as standard deviations for these values measured for different patients, are known. For example, the standard variation of T1 or T2 decays, respectively, lies within the range of 20% to 30% for measurements on different patients. This standard deviation is less than the standard deviation that would be obtained by measuring the signal-to-noise ratio at an arbitrary position within the patient image data depicting an arbitrary type of tissue. For a measurement in an arbitrary position, no reference information enabling the assessment of the performance of the magnetic resonance imaging apparatus and/or the determination of the imaging parameters may be provided. Criteria for selecting landmarks for which reference values are to be provided in the database may include a standard deviation of measured magnetic resonance signals for a representative population of patients to be below a certain threshold and/or criteria relating to their detectability in patient image data acquired using the predetermined second magnetic resonance sequence.

In one embodiment, a magnetic resonance sequence that is independent of the individual patient (e.g., a pre-scan sequence or a localizer sequence) is used as the second magnetic resonance sequence. By the usage of a sequence that is conducted independently on the patient, which provides that no parameters of the sequence are adapted to the patient, the comparability of different landmark-specific signal-to-noise ratios may be enabled since the patient image data is obtained using an equal or a similar sequence. For example, a pre-scan sequence or a localizer sequence that is conducted at the beginning of every examination process with identical or nearly identical parameters may be used. In this manner, the determination of the imaging quality information is integrated into the examination process without the need to add further measurements regarding the signal information. In this respect, a noise measurement may also already be performed for other purposes. In one embodiment, the measurement sequence protocol of the second magnetic resonance sequence is kept constant or close to constant for different patient examination processes.

In one embodiment, a magnetic resonance sequence without excitation pulses is used as first magnetic resonance sequence. The noise information may be measured using this first magnetic resonance sequence without excitation pulses and/or by performing a read-out with no gradients applied. By using a sequence without excitation, no magnetic resonance signal is created and only the noise level that may include, for example, stochastic noise and/or noise arising from the setup is measured.

In an embodiment, the first magnetic sequence corresponds to the second magnetic resonance sequence without excitation pulses. For example, the noise measurement is conducted using the first magnetic resonance sequence that is the same as the second magnetic resonance sequence except for the excitation pulses. For example, all parameters of the first magnetic resonance sequence, which are not related to the generation of excitation pulses, may be equal or similar to the parameters of the second magnetic resonance sequence. In this way, the noise information is obtained by a first magnetic resonance sequence that is similar to the second magnetic resonance sequence used for the measuring of the patient image data.

Embodiments may provide that reference information including a reference signal-to-noise ratio is used, where the reference signal-to-noise ratio is calculated and/or empirically determined and/or determined based on previously measured landmark-specific signal-to-noise ratios stored in the database (e.g., as a mean or a weighted average of previously measured landmark-specific signal-to-noise-ratios). For the assessment of the performance of the magnetic resonance imaging apparatus and/or for the determination of the imaging parameters, the imaging quality information determined from the landmark-specific signal-to-noise ratio of the current examination process is compared to the statistically and/or empirically and/or theoretically determined reference information from the database.

The reference information may include, for example, a reference signal-to-noise ratio that may be calculated, for example, using simulation algorithms, theoretical frameworks, and/or the like, or the reference signal-to-noise ratio may be empirically determined from a number of previously conducted calibration measurements (e.g., directly after the magnetic resonance apparatus has been installed). In one embodiment, the reference information includes landmark-specific signal-to-noise ratios previously measured in actual examination processes and/or at least one mean or weighted average of such previously measured landmark-specific signal-to-noise ratios.

An embodiment of the method may provide that the landmark-specific signal-to-noise ratio is determined by averaging a signal intensity within a defined image region including or comprised by the landmark. For example, for each landmark, a rectangular or quadratic image region, which includes the landmark, may be used for the determination of the signal intensity. For example, the signal intensity within a defined number of pixels or voxels of the patient image data including the landmark may be averaged by calculating, for example, the mean of the signal intensity of each pixel or voxel of the image region and by using the mean as the signal intensity of the landmark for the determination of the landmark-specific signal-to-noise ratio.

Embodiments may provide that the imaging quality information includes an average of the current landmark-specific signal-to-noise ratio and previously measured landmark-specific signal-to-noise ratios stored in the database (e.g., as a mean and/or a sliding-window average and/or a weighted average of the previously measured landmark-specific signal-to-noise ratios). For example, a defined number of previously measured landmark-specific signal-to-noise ratios as well as the signal-to-noise ratio of the current examination process may be used to determine an average of the landmark-specific signal-to-noise ratios as imaging quality information to allow a more robust estimation. In one embodiment, a weighted average that weights recently acquired signal-to-noise ratios higher than older signal-to-noise ratios may be used.

The average may also be a sliding-window average based on a defined number of previously measured landmark-specific signal-to-noise ratios and the current landmark-specific signal-to-noise ratio. The sliding window average may be determined, for example, from the current landmark-specific signal-to-noise ratio and nine previously determined landmark-specific signal-to-noise ratios from the database, which were determined during the nine preceding examination processes. Also, a weighting of the previously measured signal-to-noise ratio may be conducted while determining the sliding-window average, so that, for example, recently determined signal-to-noise ratios are weighted higher than older ones.

The determined imaging quality information is compared to the reference information to assess the performance of the magnetic resonance imaging apparatus. In one embodiment, the comparison may include the determination of a trend information that describes the behaviour of the imaging quality information (e.g., landmark-specific signal-to-noise ratios) over all or a defined number of previously performed measurements. For that purpose, imaging quality information including the currently determined landmark-specific signal-to-noise ratio and a reference information including previously determined landmark-specific signal-to-noise ratios may be compared. A decreasing landmark-specific signal-to-noise ratio may be an indicator that a calibration of the magnetic resonance imaging apparatus should be performed. To reduce the influence of measurement errors or statistical fluctuations, statistical averages (e.g., imaging quality information including an average of the currently determined landmark-specific signal-to-noise ratio and a defined number of previously determined landmark-specific signal-to-noise ratios and corresponding averages for earlier time intervals as reference information) may be used. For example, an average of the current and nine preceding signal-to-noise ratios may be compared to the reference information, which includes, for example, a threshold signal-to-noise ratio or an average of fifty previously determined signal-to-noise ratios for the specific landmark, so that a decrease and/or a deterioration of the landmark-specific signal-to-noise ratio may be determined enabling, for example, the assessment of the performance of the magnetic resonance imaging apparatus.

A notification to an operator of the magnetic resonance imaging apparatus is generated if the performance of the magnetic resonance imaging apparatus assessed from the comparison of the imaging quality information and the reference information meets at least one notification criterion. As notification criterion, for example, a minimally allowed landmark-specific signal-to-noise ratio may be used as a threshold. It is also possible that a certain decrease rate in signal-to-noise ratios within a defined number of previously performed measurements is used as a notification criterion, which enables the detection of a decreasing or deteriorating performance before the landmark-specific signal-to-noise ratio drops below the threshold. For example, a prediction of when the threshold is reached may be determined depending on trend information. A notification to an operator of the magnetic resonance imaging apparatus may be generated by notifying the operator on a signal-to-noise ratio below the threshold or on an on-going decrease of the signal-to-noise ratios within a defined number of last measurements or within a defined time period or a soon to occur drop of the signal-to-noise ratio below the threshold or the like.

In one embodiment, a contrast-to-noise ratio for a pair of landmarks is determined as imaging quality information, where the reference information includes a reference contrast-to-noise ratio. The contrast-to-noise ratio may be calculated based on the determined landmark-specific signal-to-noise ratios for the pair of landmarks. In one embodiment, two landmarks of different tissue type/composition are used for the determination of the contrast-to-noise ratio. For the assessment of the performance of the magnetic imaging apparatus and/or the determination of the imaging parameters, the imaging quality information including a contrast-to-noise ratio may be compared to reference information that also includes a reference contrast-to-noise ratio. Thus, in the imaging quality information, the contrast-to-noise ratio may be used as a surrogate for the signal-to-noise ratio itself or additionally to them, so that all remarks above also apply.

In an addition to the method of one or more of the present embodiments, which may also be used independently of assessing the magnetic resonance imaging apparatus, an optimization of the imaging parameters of at least one imaging sequence of the current examination process may be conducted based on the contrast-to-noise ratio of at least one pair of landmarks. Based on the determined contrast-to-noise ratio from the landmark-specific signal-to-noise ratios of a pair of landmarks, an optimization of the imaging sequence and hence the current examination process may be performed by determining imaging parameters for the imaging sequence of the current examination process. The optimization target may be a maximization of the contrast-to-noise ratio between two different landmarks, which facilitates distinguishing, for example, the tissue of the first landmark from the tissue of second landmark of the pair of landmarks.

Additionally, it may be provided that, for the optimization of the imaging parameters, at least one modified imaging sequence differing from the imaging sequence in at least one imaging parameter value is used, where an optimization for the at least one imaging parameter value is conducted based on a comparison of the contrast-to-noise ratio of the imaging sequence and a contrast-to-noise ratio determined from the at least one modified imaging sequence. By using the modified imaging sequence, which differs from the imaging sequence in at least one imaging parameter, also a contrast-to-noise ratio for the modified imaging sequence may be determined from landmark-specific signal-to-noise ratios of a pair of landmarks, so that from a comparison of the contrast-to-noise ratios of the imaging sequence and the modified sequence, an optimization of the imaging parameters with respect to the contrast-to-noise ratio may be performed. For example, the optimization of the imaging parameters may target a maximization of the contrast-to-noise ratio.

In an embodiment, an optimized sequence is generated by multiple usage of differently modified imaging sequences and an optimization of the contrast-to-noise ratio in dependence of the at least one imaging parameter. This allows performing a step-by-step optimization procedure, which is based on the evaluation of the contrast-to-noise ratios for the different modified sequences including at least one different imaging parameter. By doing so, the influence of the parameter on the contrast-to-noise ratio may be determined, and an optimized parameter value for each imaging parameter may be found, leading to an optimized sequence with an optimized or optimal contrast-to-noise ratio. An optimized or optimal contrast-to-noise ratio may be, for example, a high contrast-to-noise ratio between a pair of landmarks.

A magnetic resonance imaging apparatus according to one or more of the present embodiments includes a control unit configured to perform a method according to one or more of the present embodiments. All remarks regarding the method also apply to the magnetic resonance imaging apparatus. The control unit may include, for example, a noise measurement unit configured to measure the noise information by using the first magnetic resonance sequence. The control unit may also include a patient data acquisition unit configured to acquire the patient image data using the second magnetic resonance sequence. The control unit may include a landmark localization unit configured to localize at least one anatomical landmark in the patient image data. The control unit may also include a signal-to-noise ratio determination and comparison unit that is configured to determine the landmark-specific signal-to-noise ratio as well as the imaging quality information based on the landmark-specific signal-to-noise ratio, and perform a comparison to the reference information. The reference information may be stored in a database of the magnetic resonance imaging apparatus or in an external database, where the control unit is configured to communicate with the database.

A computer program according to one or more of the present embodiments may include instructions that, when the program is executed by a computer, cause the computer to carry out a method according to the present embodiments. The computer may be the control unit of the magnetic resonance imaging apparatus according to the present embodiments. It is also possible that the computer is an external computing unit that communicates with the magnetic resonance imaging apparatus.

An electronically readable storage medium (e.g., a non-transitory computer-readable storage medium) according to one or more of the present embodiments has a computer program (e.g., with instructions) stored thereon. The instructions are executable by the control unit to execute a method of one or more of the present embodiments.

### BRIEF DESCRIPTION OF THE DRAWINGS

FIG. 1 shows a schematic diagram of one embodiment of a method; and

FIG. 2 shows a schematic view of one embodiment of a magnetic resonance imaging apparatus.

### DETAILED DESCRIPTION

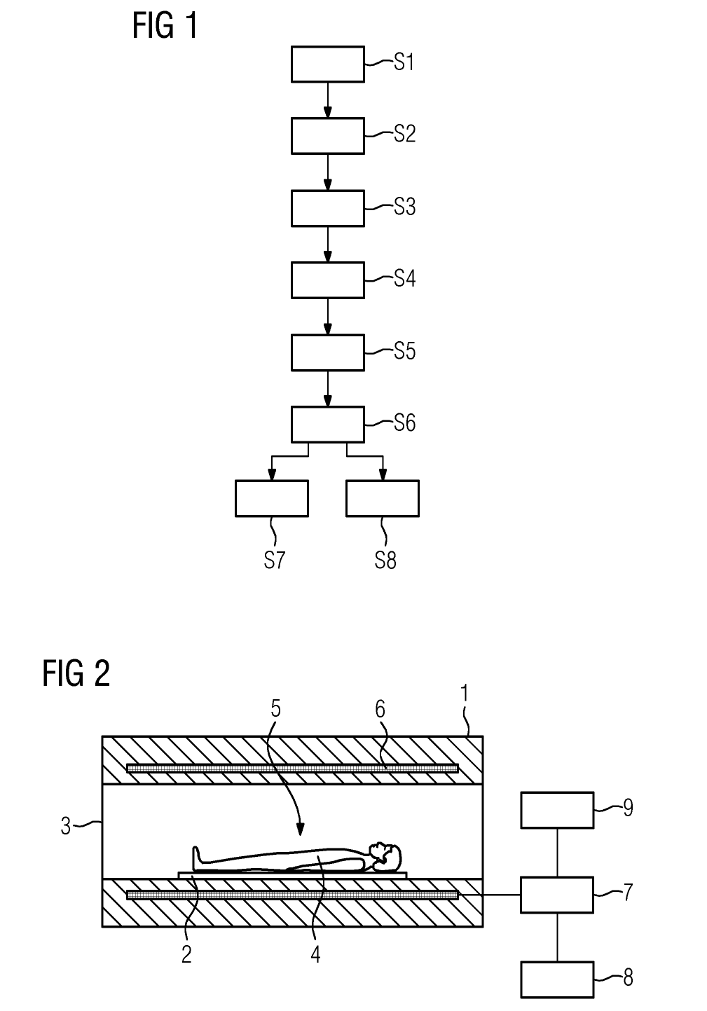

In FIG. 1, a flow diagram of a method according to an embodiment is shown. The acts are numbered as follows:

| | |
|---|---|
| S1 | Start |
| S2 | Measuring the noise information using the first magnetic |
| | resonance sequence |
| S3 | Acquisition of patient image data using the second magnetic |
| | resonance sequence |
| S4 | Landmark localization |
| S5 | Determination of the signal-to-noise ratio |
| S6 | Determination of the imaging quality information and |
| | comparison with the reference information |
| S7 | Assessment of performance and notification |
| S8 | Determination of imaging parameters and optimization of |
| | imaging sequence |

The method according to the one or more of the present embodiments begins in act S1. In one embodiment, the method is conducted at the beginning of an examination process of a patient. The patient is therefore positioned at least partly within an imaging volume of the magnetic resonance imaging apparatus.

In act S2, a noise information describing the general noise properties of the magnetic resonance apparatus in the imaging volume is measured using a predetermined first magnetic resonance sequence. This first magnetic resonance sequence does not contain any excitation pulses and is used only for measuring or recording of the noise level of at least one coil element used for imaging. As coil elements, for example, the coil elements of a body coil or another radio-frequency coil of the magnetic resonance imaging apparatus may be used. In one embodiment, additional coil elements are used, or a coil element or a plurality of coil elements not including the body coil is used (e.g., coil elements of integrated, local coils of the magnetic resonance imaging apparatus). The noise level of the at least one coil element may be determined every time that a new coil configuration is selected for imaging or that the table, on which the patient is positioned, is moved. Based on the results of the noise measurement, for each channel or for each coil, respectively, a complex vector N_{cHA} of length k is obtained as:

n_{cHA}=(a₀+b₀i; a₁+b₁i; . . . ; aₖ+bₖi),

where a and b denote each of the k complex measurement values. For each channel or for each coil, the noise level may be calculated as

$${noise\_level}_{CHA} = \frac{{\sum }_{n = 1}^{k} {a}_{n}^{2} + {b}_{n}^{2}}{k} .$$

This noise level depends on one or more protocol parameters like coil gain and bandwidth, which may also be considered when using this information (e.g., by the usage of at least one corresponding correction factor). A general noise level denoted as noise_level may be obtained by combining the noise levels of all relevant n channels (each corresponding to a coil element) as:

noise_level=[noise_level_{CHA1}+noise_level_{CHA2}+, . . . , noise_level_{CHAn}],

where a combined noise vector denoted combined noise vector may be calculated as:

combined_noise_vector=noise_level*noise_decorr_matrix,

where noise decorr matrix denotes a noise decorrelation matrix. A scalar combined noise level denoted combined_noise_level may be calculated as:

$$combined\_noise \_level = \sqrt{{noise\_level}_{CHA 1}^{2} + {noise\_level}_{CHA 2}^{2} + , … , {noise\_level}_{CHAn}^{2}} ,$$

where the combined noise level describes the general noise properties of the magnetic resonance apparatus in the imaging volume when using the at least one coil element. The combined noise vector and/or the combined noise level may be used as noise information. It is also possible that another type of noise information and/or another way of calculating a combined noise vector or a combined noise level may be used within the scope of the present embodiments. A combined noise level describes the average noise level within the imaging volume of the magnetic resonance imaging apparatus.

In act S3 of the method, patient image data is acquired in at least one imaging region of the patient using a predetermined second magnetic resonance sequence. As the second magnetic resonance sequence, a pre-scan sequence or a localizer sequence executed in the beginning of the examination process may be used. The patient image data contains at least one patient image of the imaging region of the patient. In one embodiment, a first magnetic resonance sequence is used in act S2 resembling the second magnetic resonance sequence except excitation pulses. In this case, the noise information may be determined with equal or close to equal parameters to the acquisition of the patient image data in act S3 except for the excitation. The order of act S2 and S3 is arbitrary; it is therefore possible that the patient image data is acquired before the noise measurement is performed.

In act S4, at least one anatomical landmark and/or at least one pair of anatomical landmarks of a group of predetermined anatomical landmarks is localized in the patient image by a landmark detection algorithm. The calculation of the algorithm may be performed by a control unit of the magnetic resonance imaging apparatus or by a landmark localizing unit of a control unit of the magnetic resonance imaging apparatus. For each of the predetermined anatomical landmarks, reference information is provided in a database. The database may be part of the magnetic resonance imaging apparatus, or the database may be an external database that is established to communicate with a control unit of the magnetic resonance imaging apparatus.

In act S5, from the patient image data, a signal intensity of the landmark is obtained. The signal intensity may be calculated, for example, for an image area including or comprised by the landmark. Assuming, for example, a punctate landmark, the signal intensity may be calculated, for example, as an average of the signal intensities in pixels or voxels around the pixel or voxel of the punctate landmark. For example, a rectangular area of n by m pixels or voxels around the punctual landmark may be used to calculate the signal-to-noise ratio of the i-th landmark SNR_landmark[i] as:

$$SNR\_landmark [ i ] = \frac{{\sum }_{x = posx\_landmark [ i ] - n}^{posx\_landmark [ i ] + n} {\sum }_{y = posy\_landmark [ i ] + m}^{posx\_landmark [ i ] - m} Image ( x , y )}{combined\_noise \_level} ,$$

where the nominator denotes the signal intensity of each pixel or voxel within the rectangular n-times m image area Image(x,y) of the patient image and where the denominator contains the combined noise level as noise information to obtain the signal-to-noise ratio.

In act S6, an imaging quality information is determined from the landmark-specific signal-to-noise ratio, where the imaging quality information is compared to the corresponding reference information for the respective landmark, which is stored in the database. In one embodiment, the landmark-specific signal-to-noise ratio is used as imaging quality information, and the imaging quality information is compared to reference information, which also includes a reference signal-to-noise ratio, for example, as a threshold or as a minimally allowed signal-to-noise ratio. This may be obtained by the imaging procedure. In one embodiment, the imaging quality information is based on the landmark-specific signal-to-noise ratio determined in act S5 as well as on previously measured and determined landmark-specific signal-to-noise ratios of the respective landmark that have been stored in the database. For example, the imaging quality information may contain an average of the currently determined and several precedingly determined landmark-specific-to-noise ratios. This average may be a weighted average and/or a sliding-window average that only refers to the currently determined signal-to-noise ratio and a certain number of previously determined signal-to-noise ratios. For example, the landmark-specific signal-to-noise ratios obtained during the current examination as well as during the last nine examination processes for the respective landmark performed with the magnetic resonance imaging apparatus may be evaluated. Additionally, a weighting of the different signal-to-noise ratios may be provided, so that, for example, the more recently acquired landmark-specific signal-to-noise ratios are weighted higher than the older landmark-specific signal-to-noise ratios. Any other number of signal-to-noise ratios may be evaluated, and/or another method of weighting may be used.

The reference information may include a threshold signal-to-noise ratio that describes a minimally allowed landmark-specific signal-to-noise ratio. The imaging quality information including the currently determined signal-to-noise ratio and/or an average of the currently determined signal-to-noise ratio and several previously determined signal-to-noise ratios may be compared to this reference information to determine whether the currently determined signal-to-noise ratio is above the threshold signal-to-noise ratio.

In one embodiment, the reference information includes several previously determined landmark-specific signal-to-noise ratios or averages thereof. Trend information describing a trend of the currently determined signal-to-noise ratio or an average containing the currently determined signal-to-noise ratio and a predefined number of previously recorded landmark-specific signal-to-noise ratios or averages thereof is determined by a comparison of the imaging quality information and the reference information. The reference information may include a mean or a weighted average of the previously measured landmark-specific signal-to-noise ratios.

Depending on the comparison between the imaging quality information and the reference information in act S6, an assessment of a performance of the magnetic resonance imaging apparatus in act S7 and/or an optimization of imaging parameters in act S8 may be performed.

For the assessment of the performance of the magnetic resonance imaging apparatus in act S7, an operator of the magnetic resonance imaging apparatus may be notified if the comparison between imaging quality information and reference information meets a notification criterion. The performance may be assessed by evaluating whether the currently determined landmark specific signal-to-noise ratio (or an average containing the currently determined landmark specific signal-to-noise ratio) is below a threshold reference signal-to-noise ratio. If this is the case, a notification on the necessity of a calibration of the magnetic resonance imaging apparatus may be generated and used to inform an operator of the magnetic resonance imaging apparatus. The notification may be output as a text on a screen of the magnetic resonance imaging apparatus and/or as an audible signal. Such a notification replaces the necessity for regular periodic calibration procedures, as a notification to an operator occurs when an insufficient signal-to-noise ratio is obtained.

An assessment of the performance is also possible by evaluating a trend within the landmark-specific signal-to-noise ratios during the comparison of the imaging quality information and the reference information. For this purpose, imaging quality information including the currently measured landmark-specific signal-to-noise ratio and a reference information including a plurality of previously measured signal-to-noise ratios may be used. From the currently measured landmark-specific signal-to-noise ratio or an average containing the currently measured landmark-specific signal-to-noise ratio and a number of previously measured signal-to-noise ratios or at least one average thereof, a trend information may be determined, so that, for example, a decrease or a deterioration of the landmark-specific signal-to-noise ratio during the last examination procedures may be detected. Additionally or alternatively, as described, a reference information including at least one mean or weighted average of the previously measured landmark-specific signal-to-noise ratios may be used for the determination of the trend information.

Additionally or alternatively to the assessment of the performance of the magnetic resonance imaging apparatus, in act S8, a determination of imaging parameters for an imaging sequence of the current examination process may also be provided.

As the noise information has already been determined and the determination of a current landmark-specific signal-to-noise ratio has been established, the information and acts may be further advantageously exploited. Based on obtained landmark-specific signal-to-noise ratios for a pair of landmarks, an adaption of the imaging parameters of an imaging sequence to be used in the examination process may be performed. This may include an optimization of imaging parameters.

To achieve this, a contrast-to-noise ratio CNR(A,B) of a pair of landmarks including landmark A and landmark B may be determined as:

CNR(A,B)=SNR(A)−SNR(B),

where SNR (A) and SNR (B) denote the landmark-specific signal-to-noise ratio of landmark A and landmark B, respectively, using the imaging sequence with imaging parameters that are to be optimized. Based on this contrast noise ratio, an optimization of the at least one imaging sequence of the current examination process may be performed by adapting imaging parameters.

In the optimization process, at least one imaging parameter of the imaging sequence may be varied to maximize the contrast to noise ration. Measurement of the contrast-to-noise ration may, in this process, be repeated with a modified imaging sequence modified according to the variation of the image parameters to be adapted. This allows comparison of the previously determined contrast-to-noise ratio with the current contrast to noise ratio resulting from the variation of the imaging parameters, thus assessing the variation. If a termination criterion is then fulfilled, optimization may be terminated, or a new variation of the parameters may be determined and used depending on the optimization algorithm used.

In FIG. 2, a magnetic resonance imaging apparatus 1 according to one or more of the present embodiments is shown. On a table 2 inside a bore 3 of the magnetic resonance imaging apparatus 1, a patient 4 is positioned. The patient 4 is positioned in an imaging volume 5 of the magnetic resonance imaging apparatus 1. As a coil having at least one coil element for the noise measurement using the first magnetic resonance sequence as well as for the acquisition of the patient image data using the second magnetic resonance sequence, a body coil 6 of the magnetic resonance imaging apparatus 1 may be used. The magnetic resonance imaging apparatus 1 may further include a control unit 7 that is configured to perform a method according to one or more of the present embodiments. The control unit 7 may include a noise measurement unit configured to measure the noise information by using the first magnetic resonance sequence. The control unit 7 may also include a patient data acquisition unit configured to acquire the patient image data using the second magnetic resonance sequence. The control unit 7 may also include a landmark localization unit configured to localize at least one anatomical landmark in the patient image data. The control unit 7 may include a signal-to-noise ratio determination and comparison unit that is configured to determine the landmark-specific signal-to-noise ratio as well as the imaging quality information based on the landmark-specific signal-to-noise ratio, and compare the imaging quality information to reference information stored in a database 8 of the magnetic resonance imaging apparatus 1. The magnetic resonance imaging apparatus 1 may also include a notification device 9 to display a notification in a visual or acoustic manner to an operator of the magnetic resonance imaging apparatus 1 (e.g., a loudspeaker or a screen).

Although the present invention has been described in detail with reference to the exemplary embodiments, the present invention is not limited by the disclosed examples from which the skilled person is able to derive other variations without departing from the scope of the invention.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for determining imaging quality information for a magnetic resonance imaging apparatus in an examination process of a patient positioned at least partly within an imaging volume of the magnetic resonance imaging apparatus, wherein the imaging quality information is used for assessing performance of the magnetic resonance imaging apparatus, for determining imaging parameters for an imaging protocol, or for assessing performance of the magnetic resonance imaging apparatus and for determining imaging parameters for the imaging protocol, the method comprising:
measuring noise information describing general noise properties of the magnetic resonance imaging apparatus in the imaging volume using a predetermined first magnetic resonance sequence;
acquiring patient image data in at least one imaging region of the patient using a predetermined second magnetic resonance sequence;
localizing at least one anatomical landmark of a group of predetermined anatomical landmarks in the patient image by a landmark detection algorithm, wherein for each landmark, for at least one pair of landmarks, or for each landmark and for at least one pair of landmarks, reference information regarding the imaging quality information is provided in a database;
determining a landmark-specific signal-to-noise ratio for each localized landmark from the patient image data at the landmark and the noise information; and
determining the imaging quality information from the landmark-specific signal-to-noise ratio, wherein the assessment of the performance of the magnetic resonance imaging apparatus, the determination of the imaging parameters, or the assessment of the performance of the magnetic resonance imaging apparatus and the determination of the imaging parameters are performed depending on a comparison of the imaging quality information and the corresponding reference information in the database.

2. The method of claim 1, wherein a magnetic resonance sequence that is independent of the individual patient is used as the predetermined second magnetic resonance sequence.

3. The method of claim 2, wherein a pre-scan sequence or a localizer sequence is used as the predetermined second magnetic resonance sequence.

4. The method of claim 1, wherein a magnetic resonance sequence without excitation pulses is used as the predetermined first magnetic resonance sequence.

5. The method of claim 4, wherein the first magnetic sequence corresponds to the second magnetic resonance sequence without excitation pulses.

6. The method of claim 1, wherein the reference information comprises a reference signal-to-noise ratio,
wherein the reference signal-to-noise ratio is calculated, empirically determined, determined, or any combination thereof based on previously measured landmark-specific signal-to-noise ratios stored in the database.

7. The method of claim 6, wherein the reference signal-to-noise ratio is calculated, empirically determined, determined, or any combination thereof based on previously measured landmark-specific signal-to-noise ratios stored in the database, as a mean or a weighted average of the previously measured landmark-specific signal-to-noise ratios.

8. The method of claim 1, wherein determining the landmark-specific signal-to-noise ratio comprises averaging a signal intensity within a defined image region comprising or comprised by the landmark.

9. The method of claim 1, wherein the imaging quality information comprises an average of the current landmark-specific signal-to-noise ratio and previously measured landmark-specific signal-to-noise ratios stored in the database.

10. The method of claim 9, wherein the previously measured landmark-specific signal-to-noise ratios are stored as a mean, a sliding-window average, a weighted average of the previously measured landmark-specific signal-to-noise ratios, or any combination thereof.

11. The method of claim 1, further comprising generating a notification to an operator of the magnetic resonance imaging apparatus when the performance of the magnetic resonance imaging apparatus assessed from the imaging quality information meets at least one notification criterion.

12. The method of claim 1, wherein determining the imaging quality information comprises determining a contrast-to-noise ratio for a pair of landmarks as imaging quality information, and
wherein the reference information comprises a reference contrast-to-noise ratio.

13. The method of claim 1, further comprising performing an optimization of at least one imaging parameter of at least one imaging sequence of a current examination process based on a contrast-to-noise ratio for a pair of landmarks.

14. The method of claim 13, wherein performing the optimization of the at least one imaging parameter comprises using at least one modified imaging sequence differing from the imaging sequence in at least one imaging parameter value, and
wherein an optimization for the at least one imaging parameter value is performed based on a comparison of the contrast-to-noise ratio of the imaging sequence and a contrast-to-noise ratio determined from the at least one modified imaging sequence.

15. The method of claim 14, wherein an optimized imaging sequence is generated by multiple usage of differently modified imaging protocols and an optimization of the contrast-to-noise ratio in dependence of the at least one imaging parameter.

16. A magnetic resonance imaging apparatus comprising:
a controller configured to determine imaging quality information for a magnetic resonance imaging apparatus in an examination process of a patient positioned at least partly within an imaging volume of the magnetic resonance imaging apparatus, wherein the imaging quality information is used for assessing performance of the magnetic resonance imaging apparatus, for determining imaging parameters for an imaging protocol, or for assessing performance of the magnetic resonance imaging apparatus and for determining imaging parameters for the imaging protocol, the determination of the imaging quality information comprising:
measurement of noise information describing general noise properties of the magnetic resonance imaging apparatus in the imaging volume using a predetermined first magnetic resonance sequence;
acquisition of patient image data in at least one imaging region of the patient using a predetermined second magnetic resonance sequence;
localization of at least one anatomical landmark of a group of predetermined anatomical landmarks in the patient image by a landmark detection algorithm, wherein for each landmark, for at least one pair of landmarks, or for each landmark and for at least one pair of landmarks, reference information regarding the imaging quality information is provided in a database;
determination of a landmark-specific signal-to-noise ratio for each localized landmark from the patient image data at the landmark and the noise information; and
determination of the imaging quality information from the landmark-specific signal-to-noise ratio, wherein the assessment of the performance of the magnetic resonance imaging apparatus, the determination of the imaging parameters, or the assessment of the performance of the magnetic resonance imaging apparatus and the determination of the imaging parameters are performed depending on a comparison of the imaging quality information and the corresponding reference information in the database.

17. In a non-transitory computer-readable storage medium that stores instructions executable by one or more processors to determine imaging quality information for a magnetic resonance imaging apparatus in an examination process of a patient positioned at least partly within an imaging volume of the magnetic resonance imaging apparatus, wherein the imaging quality information is used for assessing performance of the magnetic resonance imaging apparatus, for determining imaging parameters for an imaging protocol, or for assessing performance of the magnetic resonance imaging apparatus and for determining imaging parameters for the imaging protocol, the instructions comprising:
measuring noise information describing general noise properties of the magnetic resonance imaging apparatus in the imaging volume using a predetermined first magnetic resonance sequence;
acquiring patient image data in at least one imaging region of the patient using a predetermined second magnetic resonance sequence;
localizing at least one anatomical landmark of a group of predetermined anatomical landmarks in the patient image by a landmark detection algorithm, wherein for each landmark, for at least one pair of landmarks, or for each landmark and for at least one pair of landmarks, reference information regarding the imaging quality information is provided in a database;
determining a landmark-specific signal-to-noise ratio for each localized landmark from the patient image data at the landmark and the noise information; and
determining the imaging quality information from the landmark-specific signal-to-noise ratio, wherein the assessment of the performance of the magnetic resonance imaging apparatus, the determination of the imaging parameters, or the assessment of the performance of the magnetic resonance imaging apparatus and the determination of the imaging parameters are performed depending on a comparison of the imaging quality information and the corresponding reference information in the database.
